(19) United States
(12) Patent Application Publication
(10) Pub. No.: US 2006/0217669 A1
(43) Pub. Date: Sep. 28, 2006

(54) Device for securing a blood vessel cannula to a body

(76) Inventor: Ruark Botha, Universitas Ridge (ZA)

Correspondence Address: MERCHANT & GOULD PC, P.O. BOX 2903, MINNEAPOLIS, MN, 55402-0903, US

(21) Appl. No.: 11/443,412

(22) Filed: May 30, 2006

Related U.S. Application Data
(63) Continuation-in-part of application No. 10/526,857, filed on Sep. 28, 2005.

(30) Foreign Application Priority Data
Sep. 4, 2002 (ZA) 2002/7083
Feb. 10, 2005 (ZA) 2005/1192

(51) Int. Cl. A61M 5/32

(52) U.S. Cl. 604/177; 604/180; 128/DIG. 6; 128/DIG. 26; 604/178

(57) ABSTRACT

The invention provides for a device for securing a portion of a blood vessel cannula to a human being or animal body, the device including a retaining means shaped and dimensioned to retain said cannula portion that extends exterior the body surface in a fixed position on the body; and an attachment means for removable attachment of the device to the skin thereby inhibiting movement of the cannula. The invention also provides for a method of securing a blood vessel cannula to a human being or animal body

### FIELD OF THE INVENTION

The Invention Is In the field of medical accessories, specifically devices used In the securing of a blood vessel cannula to the skin

### BACKGROUND TO THE INVENTION

Once a cannula has been successfully inserted into a blood vessel, be it intra-venous or intra-arterial, it is imperative that the cannula not be dislodged. Traditional methods require the care giver to pre-cut a plaster or use one of the newer self-adhesive plasters that require the care giver to use both hands. The inserted cannula is thus left and any movement of the patient can cause the cannula to be dislodged. It is also often difficult to secure the cannula and administration tube under one dressing and can thus lead to wastage.

A need exists for a device that can secure a cannula, is easy to use and can be applied with one hand while the other hand is used to stabilize the cannula.

### SUMMARY OF THE INVENTION

According to an aspect of the invention, there is provided a device for securing a portion of a blood vessel cannula to a human being or animal body, the device including;

a retaining means shaped and dimensioned to retain said cannula portion that extends exterior the body surface in a fixed position on the body; and

an attachment means for removable attachment of the device to the skin thereby inhibiting movement of the cannula.

The retaining means may be a deformable receptacle.

The retaining means may be a resiliently deformable receptacle.

The receptacle may include a first open position and a second closed position. When in a closed position, the receptacle may have a frusto-conical shape.

The receptacle may include a looking means configured to lock and maintain the receptacle in a closed position.

The locking means may include a clip or a clasp.

The receptacle may include a recess configured to accommodate a rim on an end of the cannula.

The receptacle may include a viewing port that permits a user to monitor for leakage or dislodgement.

The receptacle may be translucent, thereby permitting inspection of the underlying area and cannula.

The receptacle may be in the form of a clamp.

The attachment means may be in the form of a wing adjacent the receptacle. The wing may be a pliable wing.

A surface of the wing may include on at least a part thereof, an adhesive for attachment to the skin. The adhesive part may be covered by a peel off cover layer which prior to use prevents attachment to other objects. The peel off layer may include a tab configured to facilitate the removal of said layer when the device is to be used. Typically, the adhesive would be of the hypoallergenic type and water soluble.

The device may include a securing means for securing an administration tube that may be attached to the cannula. The securing means may include protrusions. The tube may be guided between said protrusions thereby to inhibit displacement thereof. Typically, the tube is guided between the protrusions before the wings are attached to the skin

It is to be appreciated from this specification, that the receptacle may be configured to accommodate a cannula attached to an administration tube where the tube has been screwed onto the cannula, or where the tube has been fitted into the cannula.

It is further to be appreciated from this specification, that the retaining means may be of unitary moulding with the cannula and may therefore not require the retaining means to be attached to the cannula after insertion of said cannula in the blood vessel.

According to another aspect of the invention, there is provided a method of securing a portion of a blood vessel cannula to a human being or animal body, said method including;

retaining said cannula portion that extends exterior the body surface in a receptacle in a fixed position on the body; and

inhibiting movement of the receptacle by removable attachment thereof to the skin.

Retaining the cannula may be by means of a resiliently deformable receptacle.

Locking the receptacle in a closed position may be by means of a clip or clasp.

Attaching the device to the skin may be by way of a pliable wing adjacent the receptacle that has on a surface of the wing an adhesive configured to be attached to the skin.

Securing an administration tube attached to the cannula may be by way of protrusions. The tube may be guided between said protrusions before attachment to the skin.

According to a further aspect of the invention, there is provided a device for inhibiting movement of a catheter or needle relative to the skin of a patient, when said catheter or needle is interconnected to an IV line or syringe by means of a hub, the device including:
a retaining means having a receiving portion for receiving at least a collar of the needle or catheter and at least a portion of the hub so that the needle or catheter collar and the hub are retained out of contact with the skin of the patient, and a base portion extending from the receiving portion; and
a flexible sheet for receiving the base portion thereon, the sheet including a transparent portion through which the needle or catheter in use enters the skin of the patient, the sheet being provided with an adhesive on its operatively lower surface to permit attachment of the sheet to the skin of the patient.

The receiving portion may be of generally frusto-conical shape and may be manufactured from any suitable synthetic plastics material.

The hub may be a conventional Luer Lock connector.

The receiving portion may be manufactured from PP Random Copolymer having the following specifications:
Bormed RF830MO
20 g/10 min (MFRO
Density: 902 kg/m³
Tensile modulus: 1150 Mpa
Hardness: 92 Rockwell
High transparency

The receiving portion may include one or more lips, ridges, or protrusions located along an inner surface thereof for supporting the needle or catheter collar and the hub thereby to retain them out of contact with the skin of the patient

The receiving portion may include a groove or recess located along an inner surface thereof for in use snugly receiving the collar of the catheter or needle and a collar of the hub.

The receiving portion may further include a flange located along an inner surface thereof to inhibit the hub from being pulled out of the receiving portion.

The base portion may be in the form of flexible wings extending from either side of the receiving portion.

The wings may be provided with a groove located in a region proximate the receiving portion, thereby permitting the wings to flex.

The base portion may be manufactured from any suitable flexible synthetic plastics material.

The retaining means may be integral with the flexible sheet. Alternatively, the retaining means may be provided with an adhesive on its operatively lower surface so that it may be attached to the flexible sheet when desired.

The flexible sheet may be manufactured from breathable urethane foam or any suitable soft, porous material.

The transparent portion may be manufactured from transparent breathable urethane film. In use, the transparent portion may seal the region of skin through which the needle or catheter enters the patient in a sterile manner, thereby inhibiting infections.

The flexible sheet may be provided with a backing to cover the adhesive when the device is not in use. The backing may be provided with one or more tabs to permit easy removal of the backing prior to use.

The adhesive may be non-allergenic and may be provided in a grid pattern on the transparent portion thereby permitting the underlying skin of the patient to be aerated.

The invention further provides a method of securing a catheter or needle, when interconnected to an IV line or syringe by means of a hub, to the skin of a patient, the method including at least the steps of:
inserting the needle or catheter into the patient so that the needle or catheter enters the skin of the patient via the transparent portion of the flexible sheet of the device as described above;
inserting at least a collar of the needle or catheter and at least a portion of the hub into the receiving portion of the device; and
securing the flexible sheet and thereby the retaining means to the skin of the patient.

### DETAILED DESCRIPTION OF DRAWINGS

The invention will now be described, by way of non-limiting example, with reference to the accompanying diagrammatic drawings wherein:

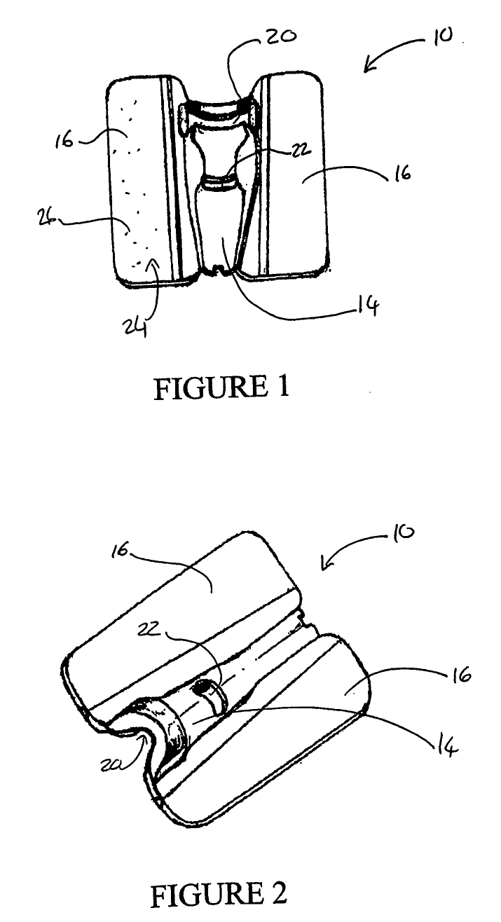

FIG. 1 shows an interior view of a device for securing a portion of a blood vessel cannula to a human being or animal body.

FIG. 2 shows a top view of the device.

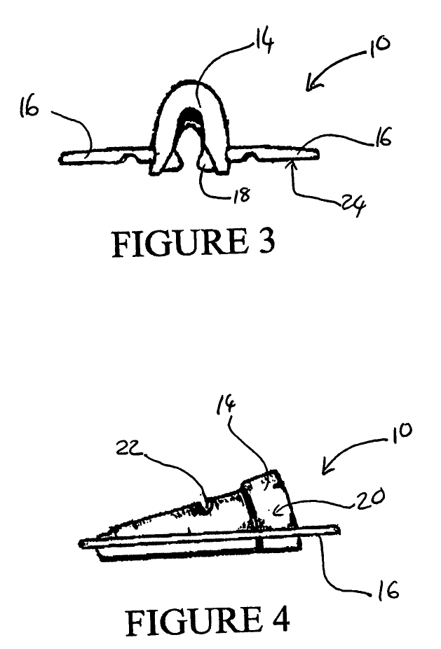

FIG. 3 shows a posterior view of the device.

FIG. 4 shows a side view of the device.

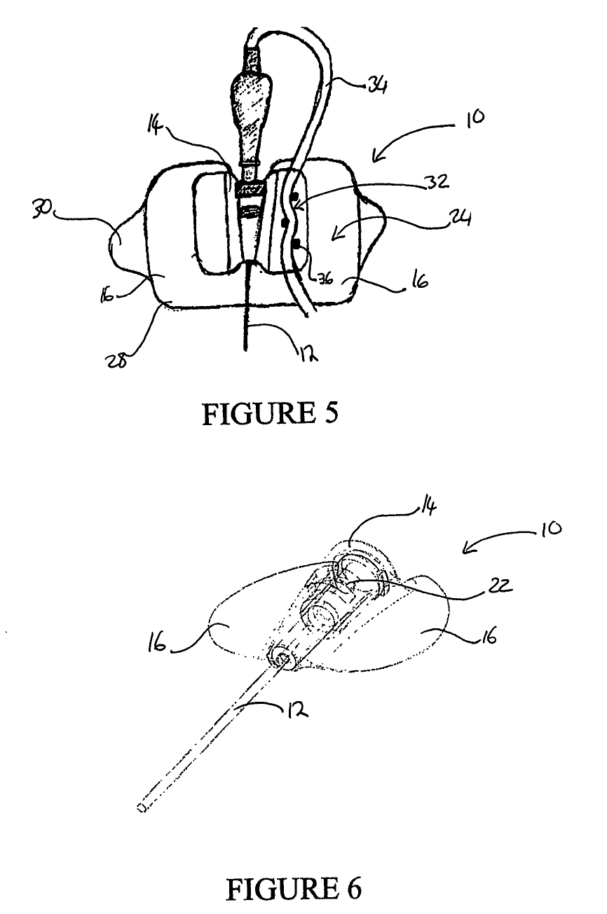

FIG. 5 shows a different embodiment of the invention.

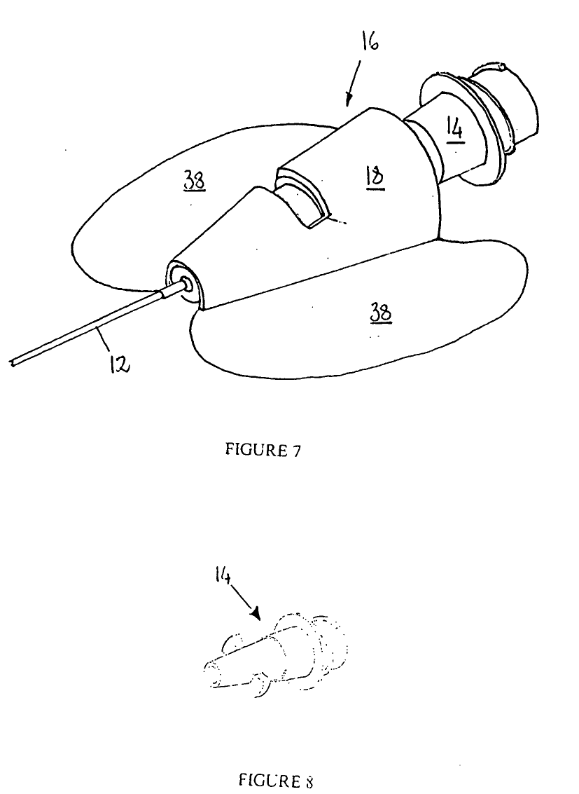

FIG. 6 shows the device as in use, with a part of the cannula being visualized through a transparent retaining means.

FIG. 7 shows a perspective view of a retaining means forming part of a device in accordance with the present invention;

FIG. 8 shows a perspective view of a hub in the form of a Luer Lock connector;

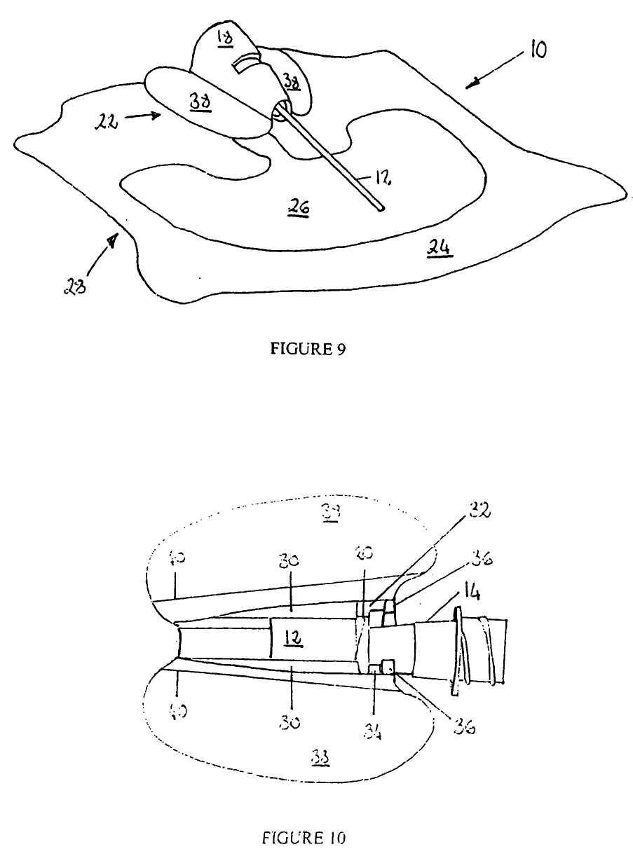

FIG. 9 shows a perspective view of a device in accordance with the present invention;

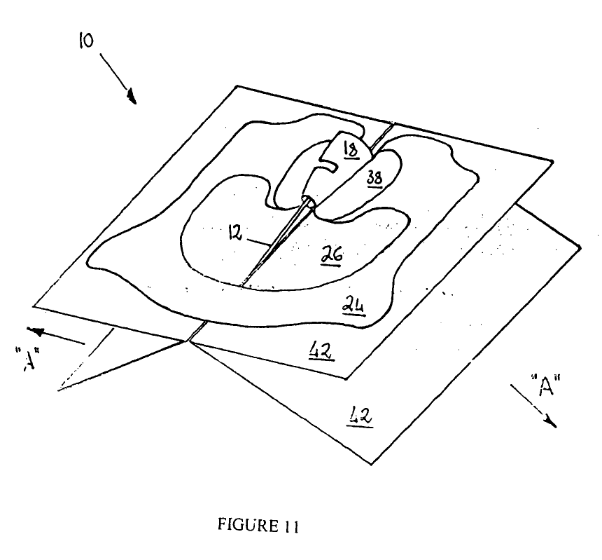

FIG. 10 shows a bottom view of the retaining means of FIG. 7 in use; and

FIG. 11 shows a perspective view of the device of FIG. 9 prior to the backing of the flexible sheet being removed.

In the drawings, reference numeral 10 generally refers to a device for securing a portion of a blood vessel cannula to a human being or animal body.

A device 10 for securing a portion of a blood vessel cannula 12 to a human being or animal being or animal body (not shown), the device including a retaining means 14 shaped and dimensioned to retain said cannula 12 portion that extends exterior the body surface in a fixed position on the body and an attachment means 16 for removable attachment of the device 10 to the skin thereby inhibiting movement of the cannula.

In a preferred embodiment, the retaining means 14 is a resiliently deformable receptacle that is capable of clamping a part of a cannula 12.

The receptacle 14 has a first open position (FIGS. 1 to 5) and a second closed position (FIG. 6). The receptacle 14 has a frusto-conical shape in the closed position.

The receptacle 14 has a clip 18. Once the receptacle 14 has been closed around a part of the cannula 12, the clip 18 keeps the receptacle in a closed position.

The receptacle 14 has a recess 20 configured to accommodate a rim (not shown) on an end of the cannula 12.

The receptacle 14 includes a viewing port 22. A connection between the cannula 12 and an administration line can be viewed through said port 22. The port 22 therefore allows a user to inspect for leakage and dislodgement.

The receptacle 14 is translucent. The translucency allows for inspection of the underlying area (not shown) and cannula 12.

The attachment means 16 are pliable wings.

A surface 24 of the wings 16 is covered by an adhesive layer 26. A tabbed cover 28 prevents attachment to other objects. The cover 28 is removed by tugging on the tab 30.

In the embodiment shown in FIG. 5, the device 10 includes a securing means 32 for securing an administration tube 34 that is attached to the cannula. The securing means 32 includes protrusions 36. The tube 24 is woven between said protrusions 36 before attaching the wing 16 to the skin.

Referring now to FIGS. 7 to 11.

The device 10 is provided to inhibit movement of a catheter 12 relative to the skin of a patient (not shown) when said catheter 12 is interconnected to an IV line (not shown) by means of a hub 14.

The device 10 includes a retaining means 16 having a receiving portion 18 for receiving at least a collar 20 of the catheter 12 and at least a portion of the hub 14 so that the catheter collar 20 and the hub 14 are retained out of contact with the skin of the patient.

The retention of the catheter collar 20 and the hub 14 out of contact with the skin of the patient serves to inhibit bruising of the patient. Bruising is further minimised by the use of soft, flexible materials in the manufacture of the device 10.

A base portion 22 extends from the receiving portion 18. The base portion 18 is mounted on a flexible sheet 24, the sheet 24 including a transparent portion 26 through which the catheter 12 in use enters the skin of the patient as shown in FIGS. 9 and 11.

The sheet 24 is provided with an adhesive on its operatively lower surface 28 to permit attachment of the sheet 24 to the skin of the patient.

The receiving portion 18 is of generally frusto-conical shape.

The hub 14 is a conventional Luer Lock connector as shown in FIG. 8.

The receiving portion 18 is manufactured from PP Random Copolymer having the following specifications:
Bormed RF830MO
20 g/10 min (MFRO
Density: 902 kg/m³
Tensile modulus: 1150 Mpa
Hardness: 92 Rockwell
High transparency

The receiving portion 18 includes two ridges 30 located along an inner surface thereof as shown in FIG. 10 for supporting the catheter collar 20 and the hub 14 thereby to retain them out of contact with the skin of the patient

The receiving portion 12 includes a recess 32 located along an inner surface thereof for in use snugly receiving the collar of the catheter 20 and a collar 34 of the hub 14.

The receiving portion 18 further includes a flange 36 located along an inner surface thereof to inhibit the hub 14 from being pulled out of the receiving portion 18.

The base portion 22 is in the form of flexible wings 38 extending from either side of the receiving portion 18.

The wings 38 are each provided with a groove 40 located in a region proximate the receiving portion 18 as shown in FIG. 10, thereby permitting the wings 38 to flex.

The base portion 22 is manufactured from any suitable flexible synthetic plastics material.

In the embodiment shown, the retaining means 16 is integral with the flexible sheet 24.

The flexible sheet 24 is manufactured from breathable urethane foam or any suitable soft, porous material.

The transparent portion 26 is manufactured from transparent breathable urethane film. In use, the transparent portion 26 seals the region of skin through which the catheter 12 enters the patient in a sterile manner, thereby inhibiting infections.

The flexible sheet 24 is provided with a backing 42 to cover the adhesive when the device 10 is not in use. The backing 42 is provided with one or more tabs (not shown) to permit easy removal of the backing prior to use as indicated by arrows “A” in FIG. 11.

The adhesive used is non-allergenic and is provided in a grid pattern on the transparent portion 26 thereby permitting the underlying skin of the patient to be aerated.

The invention extends to a method of securing the catheter 12, when interconnected to the IV line by means of the hub 14, to the skin of a patient.

The method includes at least the steps of inserting the catheter 12 into the patient so that the catheter 12 enters the skin of the patient via the transparent portion 26 of the flexible sheet 24 of the device 10, inserting at least a collar 20 of the catheter 12 and at least a portion of the hub 14 into the receiving portion 18 of the device 10 and securing the flexible sheet 24 and thereby the retaining means 16 to the skin of the patient.

The flexible sheet 24 is secured to the skin of the patient by removing the backing 42 as shown in FIG. 11.

The advantage of the device 10 of the invention lies mainly in the fact that it can easily be applied to the skin of a patient even when using only one hand and that once the device has been so attached, there is no need to disturb the catheter 12 to connect it to the IV line as this is done prior to the device 10 being attached to the patient.

1. A device for securing a portion of a blood vessel cannula to a human being or animal body, the device including; a retaining means shaped and dimensioned to retain said cannula portion that extends exterior the body surface in a fixed position on the body; and an attachment means for removable attachment of the device to the skin thereby inhibiting movement of the canula.

2. A device as claimed in claim 1, wherein the retaining means is a deformable receptacle.

3. A device as claimed in claim 2, wherein the receptacle includes a first open position and a second closed position.

4. A device as claimed in claim 3, wherein the receptacle has a FRUSTO-CONICAL shape in the closed position.

5. A device as claimed in claim 3, wherein the receptacle includes a locking means configured to lock and maintain the receptacle in a closed position.

6. A device as claimed in claim 1, wherein the retaining means includes a recess configured to accommodate a rim on an end of the canula.

7. A device as claimed in claim 1, wherein the retaining means includes a viewing port that permits a user to monitor for leakage and dislodgement.

8. A device as claimed in claim 1, wherein the retaining means is translucent and thereby permits inspection of the underlying skin area and canula.

9. A device as claimed in claim 1, wherein the retaining means is in the form of a clamp.

10. A device as claimed in claim 1, wherein the attachment means is in the form of a wing.

11. A device as claimed in claim 10, wherein a surface of the wing includes on at least a part thereof, an adhesive for attachment to the skin.

12. A device as claimed in claim 11, wherein the adhesive part is covered by a peel off cover layer.

13. A device as claimed in claim 12, wherein the peel off layer includes a tab configured to facilitate the removal of said layer before use.

14. A device as claimed in claim 1, wherein the device includes a securing means for securing an administration tube attached to the canula.

15. A device as claimed in claim 14, wherein the securing means includes protrusions that are configured to guide the tube and inhibit displacement thereof.

16. A method of securing a portion of a blood vessel cannula to a human being or animal body, said method including; retaining said cannula portion that extends exterior the body surface in a receptacle in a fixed position on the body; and inhibiting movement of the receptacle by removable attachment thereof to the skin.

17. A method of securing a BLOOD VESSEL CANNULA as claimed in claim 16, wherein the retaining of the cannula is by means of a resiliently deformable receptacle that has open and closed positions.

18. A method of securing a blood vessel cannula as claimed in claim 16, wherein the locking of the receptacle in a closed position is by means of a clip.

19. A method of securing a blood vessel cannula as claimed in claim 16, wherein the inhibiting of movement is by way of a pliable wing that has on a surface of the wing an adhesive configured to be removably attached to the skin.

20. A method of securing a blood vessel cannula as claimed in claim 16, wherein securing of an administration tube attached to the CANNULA is by way of guiding the tube between protrusions located on a surface of the device.

21. A device for inhibiting movement of a catheter or needle relative to the skin of a patient, when said catheter or needle is interconnected to an IV line or syringe by means of a hub, the device including:
a retaining means having a receiving portion for receiving at least a collar of the needle or catheter and at least a portion of the hub so that the needle or catheter collar and the hub are retained out of contact with the skin of the patient, and a base portion extending from the receiving portion; and
a flexible sheet for receiving the base portion thereon, the sheet including a transparent portion through which the needle or catheter in use enters the skin of the patient, the sheet being provided with an adhesive on its operatively lower surface to permit attachment of the sheet to the skin of the patient.

22. A device as claimed in claim 21, wherein the receiving portion is of generally frusto-conical shape.

23. A device as claimed in claim 21; wherein the hub is a conventional Luer Lock connector.

24. A device as claimed in claim 21; wherein the receiving portion is manufactured from PP Random Copolymer having the following specifications:
Bormed RF830MO
20 g/10 min (MFRO
Density: 902 kg/m³
Tensile modulus: 1150 Mpa
Hardness: 92 Rockwell
High transparency

25. A device as claimed in claim 21; wherein the receiving portion includes one or more lips, ridges, or protrusions located along an inner surface thereof for supporting the needle or catheter collar and the hub thereby to retain them out of contact with the skin of the patient

26. A device as claimed in claim 21; wherein the receiving portion includes a groove or recess located along an inner surface thereof for in use snugly receiving the collar of the catheter or needle and a collar of the hub.

27. A device as claimed in claim 21; wherein the receiving portion includes a flange located along an inner surface thereof to inhibit the hub from being pulled out of the receiving portion.

28. A device as claimed in claim 21; wherein the base portion is in the form of flexible wings extending from either side of the receiving portion.

29. A device as claimed in claim 28, wherein the wings are provided with a groove located in a region proximate the receiving portion, thereby permitting the wings to flex.

30. A device as claimed in claim 21; wherein the base portion is manufactured from any suitable flexible synthetic plastics material.

31. A device as claimed in claim 21; wherein the retaining means is integral with the flexible sheet.

32. A device as claimed in claim 21; wherein the retaining means is provided with an adhesive on its operatively lower surface so that it may be attached to the flexible sheet when desired.

33. A device as claimed in claim 21; wherein the flexible sheet is manufactured from breathable urethane foam or any suitable soft, porous material.

34. A device as claimed in claim 21; wherein the transparent portion is manufactured from transparent breathable urethane film.

35. A device as claimed in claim 21; wherein the flexible sheet is provided with a removable backing to cover the adhesive when the device is not in use.

36. A device as claimed in claim 21; wherein the adhesive is provided in a grid pattern on the transparent portion thereby permitting the underlying skin of the patient to be aerated.

37. A method of securing a catheter or needle, when interconnected to an IV line or syringe by means of a hub, to the skin of a patient, the method including at least the steps of:
inserting the needle or catheter into the patient so that the needle or catheter enters the skin of the patient via the transparent portion of the flexible sheet of the device as described above;
inserting at least a collar of the needle or catheter and at least a portion of the hub into the receiving portion of the device; and
securing the flexible sheet and thereby the retaining means to the skin of the patient.
